(19) United States
(12) Patent Application Publication
(10) Pub. No.: US 2022/0401156 A1
(43) Pub. Date: Dec. 22, 2022

(54) DEVICES, SYSTEMS, AND METHODS FOR LOCALIZING MEDICAL DEVICES WITHIN A BODY LUMEN

(71) Applicant: BOSTON SCIENTIFIC SCIMED, INC., Maple Grove, MN (US)

(72) Inventors: Daniel J. Foster, Lino Lakes, MN (US); Kyle True, Minneapolis, MN (US); Sebastian Ordas Carboni, Vadnais Heights, MN (US)

(73) Assignee: BOSTON SCIENTIFIC SCIMED, INC., Maple Grove, MN (US)

(21) Appl. No.: 17/845,675

(22) Filed: Jun. 21, 2022

Related U.S. Application Data
(60) Provisional application No. 63/213,368, filed on Jun. 22, 2021.

(51) Int. Cl. A61B 34/20; A61B 90/94; A61B 8/12; A61B 34/10

(52) U.S. Cl. CPC A61B 34/20; A61B 90/94; A61B 8/12; A61B 34/10; A61B 2090/3958

(57) ABSTRACT

The disclosure relates to localizing medical devices within a body lumen, such as for identifying the location of, or navigating to, a target tissue. Embodiments include localizing a medical device extended out of a working channel of a delivery device. The medical device may include an imaging sensor, such as for mapping the body lumen. In many embodiments, a delivery device may include one or more sensors including a first sensor to interact with a tracking system to determine the location of the delivery device within a tracking volume and a second sensor to determine the location of the medical device relative to the delivery device. In many such embodiments, a controller may determine the location of the medical device in the tracking volume based on the location of the delivery device in the tracking volume and the location of the medical device with respect to the delivery device.

### CROSS REFERENCE TO RELATED APPLICATIONS

The present application claims the benefit of and priority to U.S. Provisional Patent Application No. 63/213,368, filed Jun. 22, 2021 and titled DEVICES, SYSTEMS, AND METHODS FOR LOCALIZING MEDICAL DEVICES WITHIN A BODY LUMEN, the disclosure of which is incorporated herein by reference.

### FIELD

The present disclosure relates generally to the field of localizing a medical device within a body lumen. In particular, the present disclosure relates to devices, systems, and methods for localizing a medical device extending out of a working channel of a delivery device.

BACKGROUND

A variety of medical devices are positioned within a body lumen for diagnostic or therapeutic purposes. For example, an endoscopy is a procedure using an endoscope to look inside a body. Typically, an endoscopy procedure utilizes an elongate member (e.g., an endoscope) to access, examine, or interact with the interior of a hollow organ or cavity of a body for diagnostic or therapeutic purposes. The endoscope typically has direct visualization for viewing inside the body and/or may be equipped with ultrasound view capability. Such scopes have a profile diameter that allow the scope to be inserted into larger body lumens (e.g., gastrointestinal (GI) tract or trachea) of a certain diameter. For example, one type of endoscope, a bronchoscope can be used for visualizing the inside of the airways, up to a certain generation of airway having a diameter that can accommodate the diameter of the bronchoscope, for diagnostic and therapeutic purposes. The bronchoscope is inserted into the airways, such as through a mouth, nose, or tracheostomy. This may allow the practitioner to examine the patient's airways for abnormalities such as foreign bodies, bleeding, tumors, or inflammation. Sometimes a biopsy may be taken from inside the lungs. At a certain higher generation of airways, the diameter of the airway becomes too narrow to accommodate conventional endoscopes, which presents the challenge for improved devices having means to accurately navigate, locate, and biopsy tissue within these smaller airways or within other lumens of minimal diameter. Localizing a medical device may refer to determining a location of the medical device within a body lumen of a patient.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one aspect, the present disclosure relates to a system including a medical device, a drive cable, a marker, and a sensor. The medical device may comprise a proximal end, a distal end, and a lumen extending between the proximal and distal ends. The drive cable may have a longitudinal axis. The drive cable may be disposed in the lumen of the medical device and be configured to rotate about the longitudinal axis. The marker may be attached to the drive cable and be rotatable by the drive cable. The sensor may be configured to detect the marker rotating about the longitudinal axis.

In various embodiments, the marker comprises a magnet and poles of the magnet are aligned perpendicular to the longitudinal axis. Some embodiment comprise an imaging sensor attached to the drive cable. In some such embodiments, the imaging sensor is rotatable by the drive cable. Many embodiments include a processor communicatively coupled to the sensor and a memory communicatively coupled to the processor. The memory may comprise instructions that when executed by the processor cause the processor to measure, with the sensor, a rotating magnetic field generated by the marker attached to the drive cable. The memory may comprise instructions that when executed by the processor cause the processor to determine a location of the marker relative to the sensor based on measurement of the rotating magnetic field. The memory may comprise instructions that when executed by the processor cause the processor to determine a location of the marker in a tracking volume based on a location of the sensor in the tracking volume and the location of the marker relative to the sensor. The memory may comprise instructions that when executed by the processor cause the processor to determine the location of the marker with five degrees of freedom in the tracking volume. The memory may comprise instructions that when executed by the processor cause the processor to measure a plurality of reference magnetic fields with the sensor. The memory may comprise instructions that when executed by the processor cause the processor to determine the location of the sensor in the tracking volume based on measurement of the plurality of reference magnetic fields. The memory may comprise instructions that when executed by the processor cause the processor to determine the location of the sensor with six degrees of freedom in the tracking volume. Some embodiments include an imaging sensor attached to the drive cable, wherein the imaging sensor is rotatable by the drive cable. In some such embodiments, the memory comprises instructions that when executed by the processor cause the processor to: generate an image with the imaging sensor; and add the image to a mapping of an interior of a body based on the location of the marker in the tracking volume. In various embodiments, the image comprises a radial image. Many embodiments include a delivery device comprising a proximal end, a distal end, and a working channel extending between the proximal and distal ends. In many such embodiments, the sensor is disposed in the delivery device and the medical device is disposed in the working channel. In several embodiments, the marker comprises a magnet and the magnet is radially symmetric with the longitudinal axis of the drive cable. In various embodiments, the sensor is disposed in a tracking base, the tracking base comprising a plurality of magnetic field emitters for generating a plurality of reference magnetic fields. In many embodiments, the sensor comprises a 6 degrees of freedom (DOF) tunnel-magnetoresistance (TMR) sensor.

In another aspect, the present disclosure relates to an apparatus comprising a delivery device, a first sensor, and a second sensor. The delivery device may include an elongate member with a proximal end, a distal end, and a working channel extending between the proximal and distal ends. The first sensor may be disposed proximate the distal end of the elongate member. The first sensor may be configured to localize a portion of the delivery device in six degrees of freedom. The second sensor may be disposed proximate the working channel. The second sensor may be configured to measure one or more characteristics of a medical device disposed in the working channel of the delivery device to determine a position of the medical device relative to the delivery device.

In various embodiments, the position of the medical device relative to the delivery device comprises one or more of a linear translation and a rotational translation. In some embodiments, the one or more characteristics comprise one or more markers encoded with the position of the medical device relative to the delivery device. In some such embodiments, the one or more markers comprise one or more barcodes or one or more quick response (QR) codes. In various such embodiments, the one or more markers comprise a plurality of markers disposed along a portion of the medical device, and wherein each of the plurality of markers indicate one or more of a linear translation and a rotational translation of the medical device. In many embodiments, the one or more characteristics comprise changes in an outer surface of the medical device. In many such embodiments, the second sensor emits and detects infrared or near-infrared light bounced off the outer surface of the medical device to determine a position of the medical device relative to the delivery device. Several embodiments include a processor communicatively coupled to the first and second sensors; and a memory communicatively coupled to the processor. The memory may include instructions that when executed by the processor cause the processor to determine a location of the medical device based on localization of a portion of the delivery device in six degrees of freedom and the position of the medical device relative to the delivery device. The memory may include instructions that when executed by the processor cause the processor to measure a plurality of reference magnetic fields with the first sensor and localize the portion of the delivery device in six degrees of freedom based on measurement of the plurality of reference magnetic fields.

In yet another aspect, the present disclosure relates to a computer-implemented method. The computer-implemented method may include measuring a plurality of reference magnetic fields with a sensor. The computer-implemented method may include determining a location of the sensor in a tracking volume based on measurement of the plurality of reference magnetic fields. The computer-implemented method may include measuring a rotating magnetic field with the sensor. The computer-implemented method may include determining a location of a source of the rotating magnetic field relative to the sensor based on measurement of the rotating magnetic field. The computer-implemented method may include determining a location of the source of the rotating magnetic field in the tracking volume based on the location of the sensor in the tracking volume and the location of the source of the rotating magnetic field relative to the sensor.

In various embodiments, the computer-implemented method may include rotating a drive cable coupled to a magnet to generate the rotating magnetic field. In some embodiments, the computer-implemented method may include generating an image with an imaging sensor and adding the image to a mapping of an interior of a body based on the location of the source of the rotating magnetic field in the tracking volume. In many embodiments, the computer-implemented method may include rotating a drive cable coupled to the imaging sensor and a magnet to generate the rotating magnetic field. In many such embodiments, the image comprises a radial ultrasound image.

In yet another aspect, the present disclosure relates to an apparatus comprising a processor and a memory communicatively coupled to the processor. The memory may comprise instructions that when executed by the processor cause the processor to measure a plurality of reference magnetic fields with a sensor. The memory may include instructions that when executed by the processor cause the processor to determine a location of the sensor in a tracking volume based on measurement of the plurality of reference magnetic fields. The memory may include instructions that when executed by the processor cause the processor to measure a rotating magnetic field with the sensor. The memory may include instructions that when executed by the processor cause the processor to determine a location of a source of the rotating magnetic field relative to the sensor based on measurement of the rotating magnetic field. The memory may include instructions that when executed by the processor cause the processor to determine a location of the source of the rotating magnetic field in the tracking volume based on the location of the sensor in the tracking volume and the location of the source of the rotating magnetic field relative to the sensor.

In various embodiments, the sensor may be disposed in a delivery device and the source of the rotating magnetic field may be disposed in a medical device disposed in a working channel of the delivery device. In some embodiments, the source of the rotating magnetic field comprises a rotating permanent magnet.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. In will be appreciated that various figures included in this disclosure may omit some components, illustrate portions of some components, and/or present some components as transparent to facilitate illustration and description of components that may otherwise appear hidden. For purposes of clarity, not every component is labeled in every figure, nor is every component of each embodiment shown where illustration is not necessary to allow those of ordinary skill in the art to understand the disclosure. In the figures:

FIG. 1 illustrates various aspects of a medical system according to one or more embodiments disclosed hereby.

FIG. 2 illustrates various aspects of a tracking system according to one or more embodiments disclosed hereby.

FIG. 3 illustrates various aspects of a delivery device in conjunction with a medical device according to one or more embodiments disclosed hereby.

FIG. 4 illustrates various aspects of a sensor disposed in a delivery device according to one or more embodiments disclosed hereby.

FIG. 5 illustrates various aspects of a marker segment according to one or more embodiments disclosed hereby.

FIG. 6 illustrates various aspects of a controller for a medical system according to one or more embodiments disclosed hereby.

FIG. 7 illustrates an exemplary process flow according to one or more embodiments disclosed hereby.

FIG. 8 illustrates an exemplary computing architecture according to one or more embodiments disclosed hereby.

### DETAILED DESCRIPTION

The present disclosure relates generally to devices, systems, and methods for localizing medical devices within a body lumen, such as for identifying the location of, or navigating to, a target tissue. Some embodiments are particularly directed to localizing a medical device extended out of a working channel of a delivery device. In several such embodiments, the medical device includes an imaging sensor, such as for mapping the body lumen or for directly visualizing the anatomy. In many embodiments, a delivery device may include one or more sensors including a first sensor to interact with a tracking system to determine the location of the delivery device within a tracking volume and a second sensor to determine the location of the medical device relative to the delivery device. In many such embodiments, a controller may determine the location of the medical device in the tracking volume based on the location of the delivery device in the tracking volume and the location of the medical device with respect to the delivery device. In several such embodiments, the first and second sensors may be the same sensor, such as a 6-degrees of freedom (DOF) tunnel-magnetoresistance (TMR) sensor. In other such embodiments, the first and second sensors may be different sensors. For instance, the first sensor may be a 5-DOF location sensor, and the second sensor may be an optical or piezoelectric sensor for reading markers on the medical device. In such instances, the markers may include indications of the location of the medical device with respect to the delivery device. These and other embodiments are described and claimed.

Many challenges face medical systems for localizing medical devices within a body lumen, such as external dimension constraints for accessing and/or localizing in peripheral body lumens, where suspected cancerous nodules are commonly located. For example, delivery devices, such as endobronchial ultrasound (EBUS) scopes, are too large (e.g., outside diameter (OD) over 4 mm) to reach into peripheral portions of body lumens (e.g., peripheral airways). Further, medical devices inserted through a working channel of the delivery device are subject to external dimension constraints (e.g., OD under 2 mm) that prevent incorporation of various types and/or combinations of components and sensors. For instance, a medical device with a lumen for a biopsy needle. For instance, a 6-DOF sensor and a radial imaging sensor may not be incorporated into a medical device with a lumen for a biopsy needle without exceeding the size constraints of the working channel of the delivery device. Additionally, excluding the imaging sensor prevents real-time visualization, thereby limiting biopsy accuracy and yield. Excluding the 6-DOF sensor prevents localizing the medical device. Adding further complexity, accurate mapping techniques for a body lumen can require real-time imaging and localizing in order to associate a pre-operative image with a specific location in the body lumen. Such limitations can drastically reduce the usability and applicability of a medical device, contributing to inefficient devices with limited capabilities. It is with these considerations in mind that a variety of advantageous medical outcomes may be realized by the devices, systems, and methods of the present disclosure.

Many embodiments herein may include a medical device that includes an imaging sensor, a lumen for delivering a biopsy needle, and one or more markers configured to indicate a location of the distal end of the medical device relative to a delivery device. Several embodiments include a controller to determine the location of the medical device in a tracking volume based on the location of the medical device relative to a delivery device and a location of the delivery device in the tracking volume.

In some embodiments, an operator may insert the medical device through a working channel of a delivery device to access a target site in a peripheral body lumen. The operator may then utilize an imaging sensor in the medical device to accurately acquire a biopsy of the target site via a needle disposed in a lumen of the medical device. Further, a controller communicatively coupled to the medical device and/or the delivery device may utilize one or more markers on the medical device to localize the target site with respect to the delivery device. In various embodiments, images from the imaging sensor may be used in conjunction with locations determined from markers to map the target site and surrounding area, such as for building a three-dimensional model.

Once a biopsy sample is acquired, it may be removed and analyzed to confirm a need for treatment at the target site. When the need for treatment at the target site is confirmed, a therapeutic tool may be inserted through the working channel of the delivery device (after removal of the medical device). The location of the target site, as previously determined with the medical device, may be utilized to accurately and reliably navigate the therapeutic tool back to the target site. For instance, the therapeutic tool may include a 5-DOF sensor that can be used in conjunction with the location of the target site determined by the medical device to properly position the therapeutic tool at the target site. In other instances, a sensor with 6 DOF may be used. In various embodiments, the therapeutic tool may include one or more therapeutic devices, such as a drug delivery tool, an implant delivery tool, an ablation probe, a cryogenic probe, a microwave probe, a radio frequency (RF) probe, a laser, an irreversible electroporation (IRE) probe, and a chemical delivery tool.

One or more techniques described hereby may facilitate accurate localization of a medical device extending out of a working channel of a delivery device, leading to useful and previously capabilities, such as accurately and reliably localizing target sites and/or mapping peripheral body lumens. In these and other ways, components/techniques described here may identify methods to increase efficiency, decrease performance costs, decrease computational cost, and/or reduce resource requirements to localize and/or map portions of peripheral body lumens in an accurate, reactive, efficient, dynamic, and scalable manner, resulting in several technical effects and advantages over conventional computer technology, including increased capabilities and improved adaptability. In various embodiments, one or more of the aspects, techniques, and/or components described hereby may be implemented in a practical application via one or more computing devices, and thereby provide additional and useful functionality to the one or more computing devices, resulting in more capable, better functioning, and improved computing devices. Further, one or more of the aspects, techniques, and/or components described hereby may be utilized to improve the technical fields of acquiring biopsy samples, localizing target sites in peripheral body lumens, mapping peripheral body lumens, and localizing medical devices in peripheral body lumens.

In several embodiments, components described hereby may provide specific and particular manners of efficiently and effectively localizing a medical device in a tracking volume based on a relative location of a delivery device for the medical device in the same tracking volume. In several such embodiments, the specific and particular manners may include one or more of determining a location of the delivery device in the tracking volume, determining a location of the medical device relative to the delivery device, and determining a location of the medical device in the same tracking volume. In many embodiments, one or more of the components described hereby may be implemented as a set of rules that improve computer-related technology by allowing a function not previously performable by a computer that enables an improved technological result to be achieved. For example, the function allowed may include one or more of: determining a location of a delivery device in a tracking volume with a sensor, determining a location of the medical device relative to the delivery device with the sensor, and determining a location of the medical device in the tracking volume based on the location of the medical device relative to the delivery device and the location of the delivery device in the tracking volume. In several embodiments, the specific and particular manners and/or the function allowed may include determining a location of a delivery device in a tracking volume and determining a location of the medical device relative to the delivery device by taking magnetic field measurements with a sensor.

With general reference to notations and nomenclature used hereby, one or more portions of the detailed description which follows may be presented in terms of program procedures executed on a computer or network of computers. These procedural descriptions and representations are used by those skilled in the art to effectively convey the substances of their work to others skilled in the art. A procedure is here, and generally, conceived to be a self-consistent sequence of operations leading to a desired result. These operations are those requiring physical manipulations of physical quantities. In some embodiments, these quantities may take the form of electrical, magnetic, or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, these manipulations are often referred to in terms, such as adding or comparing, which are commonly associated with mental operations performed by a human operator. However, no such capability of a human operator is necessary, or desirable in many cases, in any of the operations described hereby that form part of one or more embodiments. Rather, these operations are machine operations. Useful machines for performing operations of various embodiments include general purpose digital computers as selectively activated or configured by a computer program stored within that is written in accordance with the teachings hereby, and/or include apparatus specially constructed for the required purpose. Various embodiments also relate to apparatus or systems for performing these operations. These apparatuses may be specially constructed for the required purpose or may include a general-purpose computer. The required structure for a variety of these machines will be apparent from the description given.

The following detailed description should be read with reference to the drawings, which depict illustrative embodiments. The present disclosure is not limited to the particular embodiments described, as such embodiments may vary. The terminology used herein is for the purpose of describing particular embodiments only and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure may be described with specific reference to medical devices and systems and procedures for treating the respiratory system, it should be appreciated that such medical devices and methods may be used to treat tissues of the abdominal cavity, digestive system, urinary tract, reproductive tract, gastrointestinal system, cardiovascular system, circulatory system, and the like.

As used herein, “proximal end” refers to the end of a device that lies closest to the user (medical professional or clinician or technician or operator or physician, etc., such terms being used interchangeably herein without intent to limit, and including automated controller systems or otherwise) along the device when introducing the device into a patient, and “distal end” refers to the end of a device or object that lies furthest from the user along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about,” in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified. The recitation of numerical ranges or values by endpoints includes all numbers within that range, including the endpoints (e.g. 1 to 5 includes 1, 1.5, 2, 2.75, 3, 3.80, 4, and 5), and fractions thereof.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

It may be understood that the disclosure included herein is exemplary and explanatory only and is not restrictive. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Although endoscopes and endoscopic systems are referenced herein, reference to endoscopes, endoscopic systems, or endoscopy should not be construed as limiting the possible applications of the disclosed aspects. For example, the disclosed aspects may be used in conjunction with duodenoscopes, bronchoscopes, ureteroscopes, colonoscopes, catheters, diagnostic or therapeutic tools or devices, or other types of medical devices or systems.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

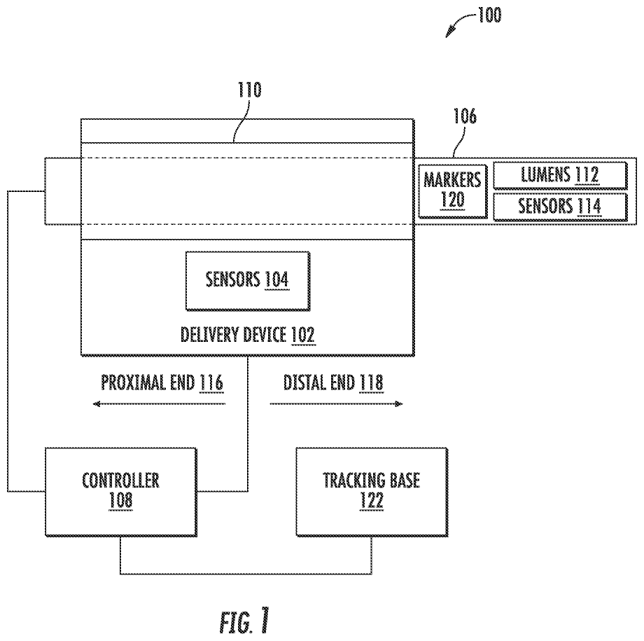

FIG. 1 illustrates various aspects of a medical system 100 according to one or more embodiments disclosed hereby. Medical system 100 may include a delivery device 102 with proximal end 116 and distal end 118, a medical device 106, a controller 108, and a tracking base 122. In various embodiments, tracking base 122 may comprise a field generator, such as a magnetic field generator. In the illustrated embodiment, the delivery device 102 includes one or more sensors 104 and a working channel 110 with medical device 106 extended therethrough. The medical device 106 includes one or more markers 120, one or more lumens 112, and one or more sensors 114. Additionally, medical device 106 may include proximal end 116 and distal end 118. In various embodiments disclosed hereby, the medical system 100 may provide functionality that enables localizing medical device 106 within a body lumen. In some embodiments, FIG. 1 may include one or more components that are the same or similar to one or more other components of the present disclosure. Further, one or more components of FIG. 1, or aspects thereof, may be incorporated into other embodiments of the present disclosure, or excluded from the described embodiments, without departing from the scope of this disclosure. For example, embodiments of medical system 100 may be directed to instructions stored on a computer-readable medium included in controller 108 without departing from the scope of this disclosure. In another example, embodiments of medical system 100 may exclude one or more of delivery device 102, medical device 106, controller 108, and tracking base 122. In yet another example, an embodiment of medical device 106 may exclude lumens 112 or sensors 114 without departing from the scope of this disclosure. Further, one or more components of other embodiments of the present disclosure, or aspects thereof, may be incorporated into one or more components of FIG. 1, without departing from the scope of this disclosure. Embodiments are not limited in this context.

Generally, delivery device 102 may include one or more sensors 104 and working channel 110. In some embodiments, delivery device 102 is a bronchoscope. In various embodiments, one or more of the sensors 104 may be operated in conjunction with tracking base 122 to determine a location of the delivery device 102 and one or more of the sensors 104 may be operated in conjunction with markers 120 to determine a location of the medical device 106 with respect to the delivery device 102. Accordingly, medical system 100 may provide functionality that enables localizing medical device 106 within a body lumen.

In many embodiments, medical device 106 includes a lumen for delivering a biopsy needle, an imaging sensor, and one or more markers configured to indicate a location of the distal end of the medical device relative to delivery device 102. In many such embodiments, delivery device 102 includes a sensor configured to read, detect, and/or measure the one or more markers 120. Several embodiments include controller 108 to determine the location of the medical device 106 in a tracking volume based on the location of the medical device 106 relative to a delivery device 102 and a location of the delivery device 102 in the tracking volume. In several such embodiments, the tracking volume may be generated by tracking base 122.

In some embodiments, an operator may insert the medical device 106 through the working channel 110 of the delivery device 102 to access a target site in a peripheral body lumen. The operator may then utilize an imaging sensor in the medical device to accurately acquire a biopsy of the target site (e.g., by providing location/position guidance). Further, controller 108 communicatively coupled to the medical device 106 and/or the delivery device 102 may utilize one or more markers 120 on the medical device to localize the target site. In various embodiments, images from the imaging sensor may be used in conjunction with locations determined from markers to map the target site and surrounding area, such as for building a three-dimensional model of the body lumen. In several embodiments, the imaging sensor may include a radial ultrasound transducer.

Once a biopsy sample is acquired, it may be removed and analyzed to confirm a need for treatment at the target site. In some embodiments, the tissue sample(s) are collected and sent to a pathology lab for diagnosis. In various embodiments, rapid on-site evaluation (ROSE) may be performed, such as with a pathologist being present to analyze the tissue sample and determine a diagnosis. In various such embodiments, real-time confirmation may enable the biopsy needle to be replaced with a therapeutic probe that can be used to treat the target site.

When the need for treatment at the target site is confirmed a therapeutic tool, such as an ablation probe, may be inserted through the working channel of the delivery device (after removal of the medical device). The location of the target site, as determined with the medical device may be utilized to accurately and reliably navigate the therapeutic tool back to the target site. For instance, the therapeutic tool may include a 6-DOF sensor, such as a 6-DOF TMR sensor, that can be used in conjunction with the location of the target site determined by the medical device to properly position the therapeutic tool at the target site. The therapeutic probe may include or utilize one or more of radio frequency (RF) waves, microwaves, cryogenic, fluids, irreversible electroporation (IRE), or other ablation modalities. In some embodiments, the therapeutic probe may be used to deliver therapeutic agents, such as chemo spheres, to the target site.

In various embodiments, medical system 100 may enable reliable and accurate access to peripheral portions of a body lumen, such as a peripheral airway. As previously mentioned, the access to peripheral portions of a body lumen may be utilized to acquire a biopsy of target tissue (e.g., a suspected cancerous nodule) and/or deliver a treatment or therapy to target tissue. Accordingly, the medical device 106 may have an outer profile small enough to fit into peripheral portions of a body lumen while still enabling one or more of real-time localization of the medical device 106 (e.g., via one or more of sensors 114), real-time imaging within the peripheral portions of the body lumen (e.g., via one or more of sensor 114), and delivery of an instrument to a target site, such as via one of lumens 112 without interfering with the real-time localization or the real-time imaging. In some embodiments, the outside diameter (OD) of the delivery device 102 may not exceed 5 mm. In other embodiments, the OD of the delivery device 102 may not exceed 4 mm. In several embodiments, the OD of the medical device 106 may not exceed 2 mm. In other embodiments, the OD of the medical device 106 may not exceed 1.8 mm.

In some embodiments, the medical device 106 may include a taper near the distal end to facilitate navigation and/or entry into small diameter body lumens, such as peripheral airways. One or more devices or embodiments herein may be sized and/or configured to be utilized for diagnostic or therapeutic purposes, such as in one or more of pulmonary, cardiac, endoscopic, and urologic applications. In various embodiments, the outer profile of the elongate member may be isodiametric with atraumatic (e.g., radiused) edges. Embodiments of medical system 100 may be utilized in a variety of applications, such as peripheral lung navigation, peripheral lung biopsy, peripheral lung ultrasound reconstruction, and peripheral lung treatment.

In several embodiments, sensors 104 may generally refer to a device that converts energy from one form into another. In many embodiments, each of the transducers may operate to convert one or more electrical signals to one or more physical quantities (e.g., energy, force, torque, light, motion, position, etcetera) and/or convert one or more physical quantities to one or more electrical signals. For example, a transducer may include one or more of an imaging sensor, a phased array sensor, a position sensor, a light emitting diode, a pressure sensor, a magnetic field sensor, a fiber-optic sensor, a piezoelectric sensor, a force sensor, or the like. In many embodiments, a magnetic field sensor may include sensors such as inductive sensing coils and/or various sensing elements such as magneto-resistive (MR) sensing elements (e.g., anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, spin Hall sensing elements, and the like), giant magneto-impedance (GMI) sensing elements, and/or flux-gate sensing elements. Many of the sensors described hereby may determine one or more of position/location in up to 6-DOF (i.e., one or more of x, y, and z measurements, and pitch, yaw, and roll angles).

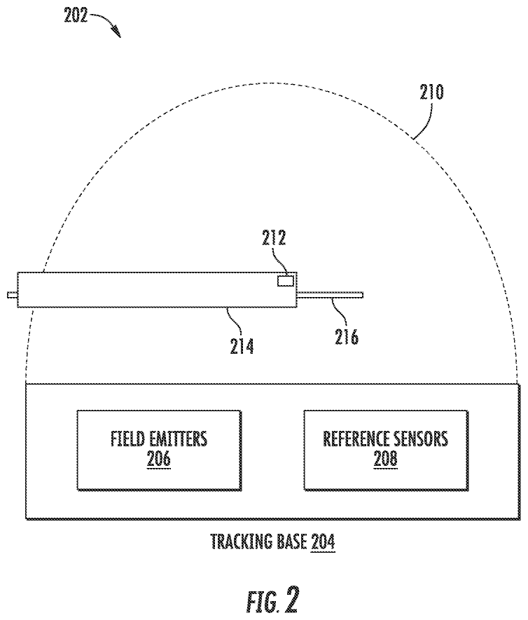

FIG. 2 illustrates various aspects of a tracking system 202 according to one or more embodiments disclosed hereby. The illustrated embodiment includes tracking base 204, delivery device 214, and medical device 216. The tracking base 204 includes field emitters 206 and reference sensors 208, and the delivery device 214 may include a magnetic field sensor 212. Tracking system 202 may be utilized to locate the magnetic field sensor 212 within tracking volume 210. In some embodiments, FIG. 2 may include one or more components that are the same or similar to one or more other components of the present disclosure. For example, tracking base 204 may be the same or similar to tracking base 122. Further, one or more components of FIG. 2, or aspects thereof, may be incorporated into other embodiments of the present disclosure, or excluded from the described embodiments, without departing from the scope of this disclosure. For example, magnetic field sensor 212 may be incorporated into delivery device 102 without departing from the scope of this disclosure. Still further, one or more components of other embodiments of the present disclosure, or aspects thereof, may be incorporated into one or more components of FIG. 2, without departing from the scope of this disclosure. For example, medical device 106 with markers 120 may be incorporated into medical device 216 without departing from the scope of this disclosure. Embodiments are not limited in this context.

Generally, tracking system 202 may operate such that the location of magnetic field sensor 212 can be determined by measuring, with magnetic field sensor 212, magnetic fields generated by each of the field emitters 206 in tracking base 204. In one or more embodiments described hereby, the location of medical device 216 (e.g., the distal end) can be determined based, at least in part, on the location of magnetic field sensor 212 within tracking volume 210. In many embodiments, a patient may be positioned within tracking volume 210. In some embodiments, one or more pre-operative scans of the patient may be overlaid with the tracking volume 210 (also referred to as registration). In various embodiments, the pre-operative scans may include or utilize magnetic resonance imaging (MRI) and/or computer tomography (CT).

For example, field emitters 206 in tracking base 204 may generate a plurality of reference magnetic fields in tracking volume 210 that may be measured with magnetic field sensor 212. In many embodiments, five or more reference magnetic fields may be measured by the sensor. For instance, tracking base 204 may generate 12 reference magnetic fields with field emitters 206 that are measured by magnetic field sensor 212. In many embodiments, a controller may calculate a vector from the magnetic field sensor 212 to each of the field emitters 206 in tracking base 204. In some embodiments, controller 608 may use the vectors combined with known geometric relationships between each of the field emitters 206 to determine the location of magnetic field sensor 212 in tracking volume 210. In some embodiments, measurements by reference sensors 208 may be utilized in conjunction with measurements by magnetic field sensor 212 to determine the location of magnetic field sensor 212 in tracking volume 210. In various embodiments, reference sensors 208 may be used for calibrating the field emitters 206 and/or the tracking volume 210. In some embodiments, reference sensors 208 may be utilized in determining the location of medical device 216. For example, reference sensors 208 may be used to measure a rotating magnetic field generated by medical device 216.

In many embodiments, magnetic field sensor 212 may comprise a 5- or 6-degree of freedom (DOF) sensor. In various embodiments, magnetic field sensor 212 may be cylindrical with a length less than 9 mm and a diameter less than 1.5 mm. In one embodiment, the magnetic field sensor 212 may include a cylindrical TMR sensor with an 8 mm length and a 0.65 mm diameter. In some embodiments, magnetic field sensor 212 may include, or be mounted to, a semi-circular, or ‘C’ shaped flexible circuit board and/or sensor. In various embodiments, the magnetic field sensor 212 may be embedded in or mounted to either the inside or the outside of the delivery device 214.

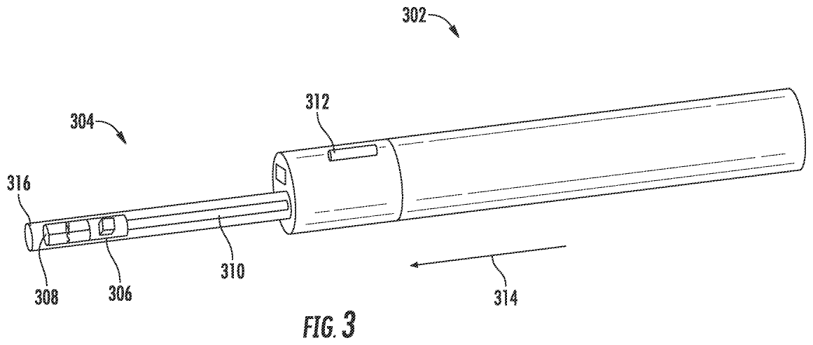

FIG. 3 illustrates various aspects of a delivery device 302 in conjunction with a medical device 304 according to one or more embodiments disclosed hereby. The illustrated embodiment includes a distal end 314 of delivery device 302 and medical device 304. The delivery device 302 includes an electromagnetic sensor 312. The medical device 106 includes a sheath 316 having an imaging sensor 306 and a marker 308 coupled to a drive cable 310 disposed therein. Embodiments disclosed hereby may include markers in a variety of shapes, forms, and materials. In the illustrated embodiment, marker 308 comprises a magnet attached to and rotatable by drive cable 310. In several embodiments, the location of marker 308 may be determined by measuring a rotating magnetic field generated by marker 308 being rotated by drive cable 310. In many embodiments, the location of marker 308 relative to electromagnetic sensor 312 may be determined based on measurements of the magnetic field generated by the marker 308. In some embodiments, FIG. 3 may include one or more components that are the same or similar to one or more other components of the present disclosure. For example, delivery device 302 may be the same or similar to delivery device 102. Further, one or more components of FIG. 3, or aspects thereof, may be incorporated into other embodiments of the present disclosure, or excluded from the described embodiments, without departing from the scope of this disclosure. For example, medical device 304 may be incorporated into medical device 604 without departing from the scope of this disclosure. Still further, one or more components of other embodiments of the present disclosure, or aspects thereof, may be incorporated into one or more components of FIG. 3 without departing from the scope of this disclosure. For example, lumens 112 of medical device 106 may be incorporated into medical device 304 without departing from the scope of this disclosure. Embodiments are not limited in this context.

In many embodiments, the marker 308 may comprise a permanent magnet that is magnetized in the cross-axis direction with respect to a longitudinal axis of drive cable 310 (i.e., the poles aligned perpendicular to the longitudinal axis). In several embodiments, marker 308 may be radially symmetric (e.g., cylinder, sphere, etcetera) with the longitudinal axis of drive cable 310. In some embodiments, the marker 308 is a permanent magnet and in other embodiments the marker 308 is an electromagnet. In various embodiments, the rotation of drive cable 310 can be combined with the cross-axis alignment of magnetic poles in marker 308 to create a rotating magnetic field with characteristics that can be measured by a sensor (e.g., electromagnetic sensor 312) to reliably determine a location of the marker 308 relative to the sensor. In several embodiments, rotating the magnetic field may assist in separating the marker 308 from background noise (e.g., magnetic field of earth).

In various embodiments, the location of the marker 308 may be determined by analyzing signals from one or more of one or more sensors in delivery device 302 (e.g., electromagnetic sensor 312), static or dynamic reference sensors placed on a patient (e.g., sensor patches), and reference sensors in the tracking system (e.g., reference sensors 208 in tracking base 204). For example, measurements of the rotating magnetic field by one or more of the sensors combined with the location, such as in 6 DOF, of the one or more sensors in a tracking volume can be utilized to determine the location of the source of the rotating magnetic field (i.e., marker 308). In some embodiments, the location of the marker 308 may be determined in 5 DOF. For example, the rotation of the marker 308 about the axis of the marker may not be determinable by measuring a magnetic field rotating about the same axis. However, in many embodiments, the rotational position of the marker 308 (e.g., the sixth degree of freedom) may be determined using other techniques. For example, one or more images generated with imaging sensor 306 may be registered to one or more pre-operative images, such as using the 5-DOF position, to determine the rotational position of the marker 308. In many embodiments, the pre-operative images may comprise CT and/or MRI images. The frequency of rotation of the marker 308 would be different that the frequency of the transmitter coils (e.g., in field emitters 206), providing a distinguishing characteristic.

In several embodiments, medical device 304 comprises a radial imaging device that utilizes drive cable 310 to rotate imaging sensor 306 and generate radial images. In several such embodiments, the imaging sensor 306 comprises an ultrasound transducer. In various embodiments, the radial images may be generated by combining a plurality of two-dimensional planar images captured by imaging sensor 306 during rotation. In some embodiments, an image processor (e.g., included in controller 108) may operate to generate radial images from a plurality of two-dimensional planar images. In many embodiments, the marker 308 is attached to a distal end of the imaging sensor 306. In other embodiments, the marker 308 may be proximal of the imaging sensor 306. One or more embodiments may include a plurality of markers 308 disposed along drive cable 310, such as both proximal and distal of imaging sensor 306. In various embodiments, data captured by imaging sensor 306 may be combined with locations determined by electromagnetic sensor 312 to map, or generate models (e.g., three-dimensional, or four-dimensional) of interior portions of a body lumen. In several embodiments, four-dimensions may refer to three-dimensions of space and one-dimension of time.

FIG. 4 illustrates various aspects of a sensor 402 disposed in a delivery device 406 according to one or more embodiments disclosed hereby. The illustrated embodiment includes distal end 410 of delivery device 406 with working channel 412 having medical device 414 with one or more markers 404 extended therethrough. In various embodiments, the sensor 402 may detect characteristics of markers 404 to determine a location of medical device 414 with respect to delivery device 406. In some embodiments, FIG. 4 may include one or more components that are the same or similar to one or more other components of the present disclosure. For example, medical device 414 may be the same or similar to medical device 106. Further, one or more components of FIG. 4, or aspects thereof, may be incorporated into other embodiments of the present disclosure, or excluded from the described embodiments, without departing from the scope of this disclosure. For example, sensor 402 may be incorporated into delivery device 214 without departing from the scope of this disclosure. Still further, one or more components of other embodiments of the present disclosure, or aspects thereof, may be incorporated into one or more components of FIG. 4 without departing from the scope of this disclosure. For example, magnetic field sensor 212 may be incorporated into delivery device 406 without departing from the scope of this disclosure. Embodiments are not limited in this context.

In some embodiments, sensor 402 is added to the working channel 412 of delivery device 406, such as embedded in a wall of working channel 412. In some such embodiments, sensor 402 may be utilized to measure one or more of the linear translation and the rotational translation of devices passed through the working channel 412. In several embodiments, the sensor 402 may detect markers 404 on medical device 414 to determine the location of medical device 414 relative to the delivery device 406. In some embodiments, the sensor 402 may utilize changes in the outer surface of devices (e.g., medical device 414) passed through the working channel 412 to determine linear and/or rotational translation. For instance, sensor 402 may emit and detect infrared or near-infrared light bounced off medical device 414 passing through working channel 412 to determine linear and/or rotational translation based on surface features. In various embodiments, sensor 402 may comprise any type of sensor capable of being utilized to localize medical device 414, such as one or more of a magnetic sensor, a piezoelectric sensor, an imaging transducer, an optical mouse sensor, and an inductive coil. In one embodiment, the sensor 402 may comprise a magneto resistive sensor. More generally, the type of sensor 402 may correspond to the type, inclusion, and/or location of markers.

The sensor 402 may be disposed along a length of the working channel 412, such as in the distal tip, along the shaft, or in the handle. In many embodiments, sensor 402 may be disposed proximate the distal end 410 of working channel 412 to limit linear and/or rotational errors. For example, linear error can result when the working channel 412 is not positioned ideally with respect to the central/neutral axis of the delivery device 406, such as due to race tracking that leads to linear translation errors at the distal end. In another example, rotational errors can result when the delivery device 406 is an imperfect torque transmitter, which can cause a rotational difference between the distal and proximal ends of the delivery device 406 when torque is applied.

In several embodiments, the sensor 402 may monitor medical devices through a wall of the working channel 412. Accordingly, in some embodiments, a viewing window may be incorporated to allow sensor 402 to monitor devices through a wall. For example, a viewing window may be utilized to replace a portion of a braided layer surrounding working channel 412. In another example, a lighter or looser braid may be utilized proximate the sensor 402. The position of sensor 402 may be determined based, at least in part, on the type, inclusion, and/or location of markers.

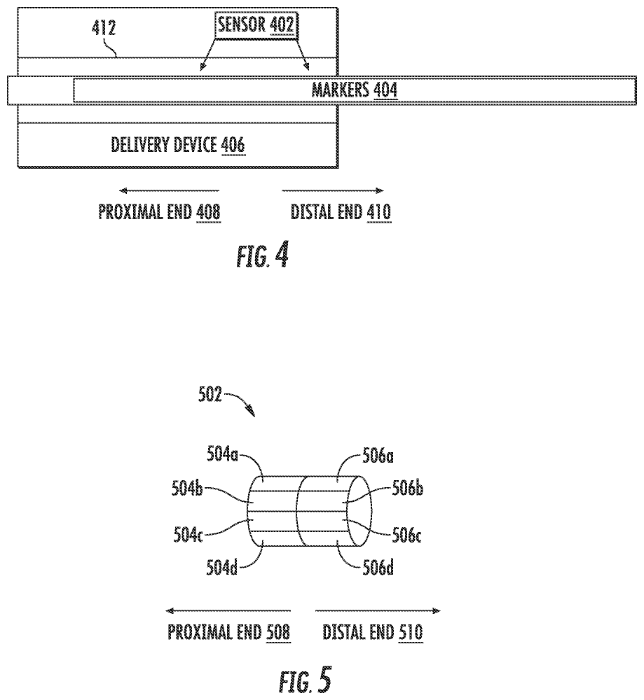

FIG. 5 illustrates various aspects of a marker segment 502 according to one or more embodiments disclosed hereby. In the illustrated embodiment, the marker segment 502 has a proximal end 508, a distal end 510, and includes markers 504a, 504b, 504c, 504d, 506a, 506b, 506c, 506d (or markers 318, 320). Embodiments disclosed hereby may include markers in a variety of shapes, forms, and materials. In many embodiments, one or more marker segments 502 may be disposed along medical device 414 (e.g., on the exterior of or embedded in). In several embodiments, one or more of the markers 318, 320 may be detected by a sensor to determine the location of a medical device relative to a delivery device. In some embodiments, FIG. 5 may include one or more components that are the same or similar to one or more other components of the present disclosure. Further, one or more components of FIG. 5, or aspects thereof, may be incorporated into other embodiments of the present disclosure, or excluded from the described embodiments, without departing from the scope of this disclosure. For example, marker segment 502 may be incorporated into markers 404 without departing from the scope of this disclosure. Still further, one or more components of other embodiments of the present disclosure, or aspects thereof, may be incorporated into one or more components of FIG. 5 without departing from the scope of this disclosure. For example, medical device 216 may be incorporated into marker segment 502 without departing from the scope of this disclosure. Embodiments are not limited in this context.

In many embodiments, markings may be placed on the shaft of the medical device 414 at known locations relative to the distal tip of the delivery device 406. In several embodiments, each marker includes an indicator, such as an encoded pattern that provides information regarding the position of the medical device relative to the delivery device. In some embodiments, each one of the markers in marker segment 502 may identify a unique linear and/or rotational displacement. For example, marker 504a may correspond to a 10 mm linear displacement and a 15-degree rotational displacement, marker 504b may correspond to a 10 mm linear displacement and a 10-degree rotational displacement, and marker 506a may correspond to a 5 mm linear displacement and a 15-degree rotational displacement.

The location, type, and/or arrangement of markers in marker segment 502 may be determined based, at least in part, on the type, inclusion, and/or location of sensors for detecting the markers (e.g., sensor 402). In various embodiments, markers may include one or more of magnetic markings (e.g., readable by a magnetic sensor), textured markings (e.g., readable by a piezoelectric sensor), markings that are only visible when illuminated with a certain wavelength of light (e.g., infrared, or ultraviolet). In one embodiment, the markings may be visible lines or patterns (e.g., barcode or quick response (QR) code) that could be detected by an optical sensor or an imaging sensor.

In one or more embodiments, the markers may comprise magnetic markings (e.g., patterns of magnetic material). For example, magnetic markings comprising a pattern of lines may be included utilizing a ferromagnetic doped polymer. In such examples, each marker in marker segment 502 may include a magnetic marking encoded with a unique linear and/or rotational position of a medical device (e.g., medical device 414). In various embodiments, a medical device may have separate rotational and linear position markers. In many embodiments, a medical device may include a plurality of marker segments 502.

In many embodiments, the magnetic markings may be read by a magneto resistive sensor. In various embodiments, the magnetic markings that are read by an inductive coil disposed around the working channel of a delivery device. In some embodiments, the marker may include one or more permanent magnets embedded or applied to the medical device. In some such embodiments, one or more magnetic sensors (such as in the delivery device) may be utilized to estimate a position and/or distance of the medical device based on the one or more static magnetic fields of the one or more permanent magnets. In several embodiments, a pattern or arrangement of permanent magnets may be utilized to create a static magnetic field with uniquely mappable features.

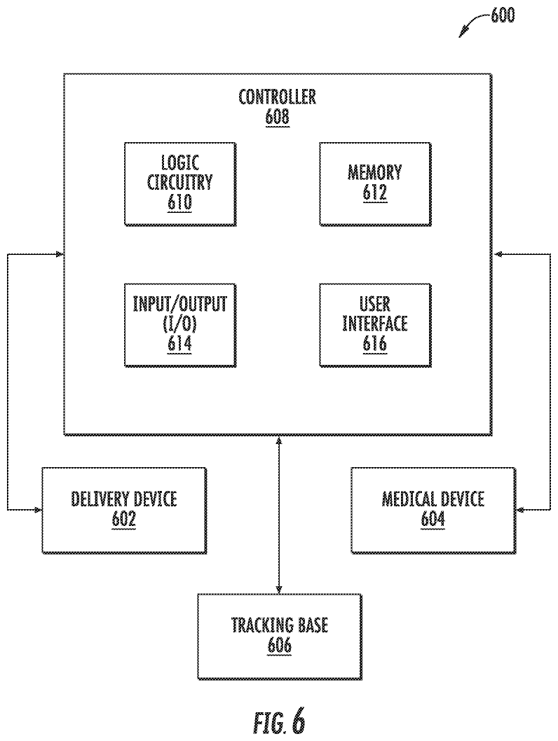

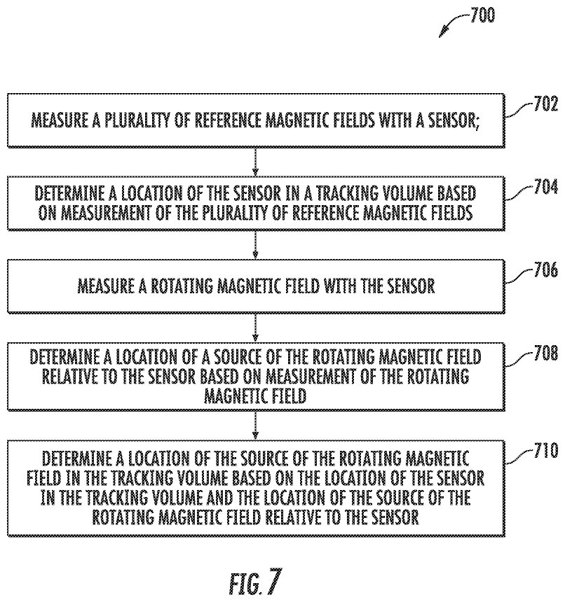

FIG. 6 illustrates various aspects of a controller 608 for a medical system 600 according to one or more embodiments disclosed hereby. In the illustrated embodiment, medical system 600 includes delivery device 602, medical device 604, tracking base 606, and controller 608. The controller 608 includes logic circuitry 610, memory 612, input/output (I/O) 614, and user interface 616. In many embodiments, controller 608 may operate to localize one or more of delivery device 602 and medical device 604 and/or map the interior of a body. In some embodiments, FIG. 6 may include one or more components that are the same or similar to one or more other components of the present disclosure. For example, medical device 604 may be the same or similar to medical device 304. Further, one or more components of FIG. 6, or aspects thereof, may be incorporated into other embodiments of the present disclosure, or excluded from the described embodiments, without departing from the scope of this disclosure. For example, tracking base 606 may be excluded from medical system 600 without departing from the scope of this disclosure. In another example, user interface 616 may be excluded from controller 608 without departing from the scope of this disclosure. Still further, one or more components of other embodiments of the present disclosure, or aspects thereof, may be incorporated into one or more components of FIG. 6, without departing from the scope of this disclosure. For example, electromagnetic sensor 312 may be incorporated into delivery device 602 without departing from the scope of this disclosure. Embodiments are not limited in this context.

In various embodiments, controller 608 may implement one or more functionalities disclosed hereby. For example, instructions stored in memory 612 may be executed by logic circuitry 610 to operate a sensor in delivery device 602 to determine a location of medical device 604. In several embodiments, logic circuitry 610 may send and receive signals from components of one or more of delivery device 602, medical device 604, and tracking base 606, via I/O 614. For example, logic circuitry 610 may determine a location of medical device 604 by utilizing a sensor in delivery device 602. In many embodiments, logic circuitry 610 may generate metadata for signals received from transducers. In many such embodiments, the metadata may correspond to signals from one or more other transducers in delivery device 602. For example, positions of the delivery device 602 indicated by a magnetic tracking sensor may be associated with images from an imaging sensor as metadata.

In many embodiments, controller 608 may track the translation and/or rotation of medical device 604 relative to delivery device 602. In many such embodiments, controller 608 may create a transformation that can be utilized to resolve the location of the medical device 604 relative to the delivery device 602. In several embodiments, location data may be utilized, such as by controller 608, for one or more of targeting, device visualization, augmented reality, mapping, and modeling. In some embodiments, three- and four-dimensional ultrasound reconstruction may be performed with an ultrasound transducer incorporated into a medical device, such as a radial ultrasound transducer. Various embodiments facilitate an accurate, intraoperative 3D image of a lesion in the coordinate space of the tracking system (e.g., tracking volume 210), such as to enable a tracked (by the tracking system) therapy delivery tool to be positioned in the correct location for optimal therapy delivery.

In several embodiments, logic circuitry 610 may perform image processing. For example, stitch a plurality of images generated by an imaging sensor (e.g., imaging sensor 306) together to create a composite image of the interior of a body. In some embodiments, logic circuitry 610 may utilize data from multiple transducers to perform mapping, such as by generating composite images. For example, position data (e.g., in 6 DOF) may be combined with images to generate composite images. In some embodiments, metadata associated with an image may indicate the position (e.g., in 6 DOF) of the imaging sensor when the imaging sensor captured the image. In various embodiments, a plurality of images may be combined to generate a radial image. In various such embodiments, a plurality of radial images may be combined to generate a composite image of an interior portion of a body.

In one or more embodiments, data from pre-operative scans may be stored in memory 612 and/or utilized by logic circuitry 610. In some embodiments, logic circuitry 602 may utilize the pre-operative data in conjunction with operative data (e.g., sensor data received from one or more of delivery device 602, medical device 604, and tracking base 606) to determine the position of delivery device 602 and medical device 604. For example, logic circuitry 610 may match landmarks identified in pre-operative images to elements in images generated via delivery device 602 to determine the position of the delivery device 602. In another example, the pre-operative images may be overlaid with a position of the patient within a tracking volume of tracking base 606 (see e.g., tracking volume 210).

In some embodiments, logic circuitry 602 may perform motion compensation. For example, logic circuitry 602 may compensate for respiratory motion. In various embodiments, motion compensation may facilitate matching operative data with pre-operative data (e.g., from computed tomography (CT) scans). For example, motion compensation may enable operative images to be overlaid on pre-operative images with reliable accuracy.

In various embodiments, logic circuitry 602 may perform one or more of object detection, distance estimation, and object classification. In various such embodiments, logic circuitry 602 may identify a mass from a plurality of ultrasound images, determine a distance to the mass based on the plurality of ultrasound images, and classify the mass based on the size, position, and distance of the mass. In several embodiments, logic circuitry 602 may utilize data from pre-operative scans to classify the mass, such as by matching the mass to a landmark identified in a pre-operative image. For example, a target nodule for biopsy located in a peripheral portion of a lung may be confirmed by comparing an object detected in an ultrasound image to one or more pre-operative images comprising the target nodule. In many embodiments, object detection and classification may be used for boundary detection, such as for identifying airway branches.

In many embodiments, logic circuitry 602 may generate a three-dimensional model of one or more portions of the interior of a body. In some embodiments, one or more techniques described hereby may be utilized to generate the three-dimensional model, such as transducer data, pre-operative data, composite images, metadata, images, positions, objects, distances, object classifications, and the like. In various embodiments, the three-dimensional model may include the position of the delivery device 602 and medical device 604.

In various embodiments, an operator may cause logic circuitry 610 to carry out various functions by providing input via user interface 616. In some embodiments, image generation may be controlled via user interface 616. In one or more embodiments, object classifications may be controlled via user interface 616. For example, a user may add or remove classifications by selecting objects in an image presented via the user interface 616. In several embodiments, logic circuitry may determine a distance between two points identified in an image presented via the user interface 616. For example, logic circuitry 602 may determine a distance between two objects selected by a user in an image. In many embodiments, output may be presented to an operator via user interface 616. For example, a three-dimensional model of a portion of the interior of a body may be presented via user interface 616.

In many embodiments, additional and/or updated functionality may be integrated into controller 608, such as by storing additional and/or updated instructions on memory 612 (e.g., as software). In one or more embodiments, controller 608 may be connected to a network (e.g., the internet, a local area network, a personal area network, or inductive coupling). In one or more such embodiments, controller 608 may be updated and/or provided with additional functionality by receiving instructions over the network.

FIG. 7 illustrates a process flow 700 according to one or more embodiments disclosed hereby. In various embodiments, one or more portions of process flow 700 may be implemented by or with components disclosed hereby. For example, blocks 702 through 710 may be implemented with controller 608 in conjunction with delivery device 302 and medical device 304. Embodiments are not limited in this context.

In the illustrated embodiment, process flow 700 may begin at block 702. At block 702 “measure a plurality of reference magnetic fields with a sensor” a plurality of reference magnetic fields may be measured with a sensor. For example, field emitters 206 in tracking base 204 may generate a plurality of reference magnetic fields in tracking volume 210 that may be measured with magnetic field sensor 212. In many embodiments, five or more reference magnetic fields may be measured by the sensor. For instance, tracking base 204 may generate 12 reference magnetic fields with field emitters 206 that are measured by magnetic field sensor 212.

Continuing to block 704 “determine a location of the sensor in a tracking volume based on measurement of the plurality of reference magnetic fields” a location of the sensor in a tracking volume may be determined based on measurement of the plurality of reference magnetic fields. For example, controller 608 may calculate a vector from the magnetic field sensor 212 to each of the field emitters 206 in tracking base 204. In some embodiments, controller 608 may use the vectors combined with known geometric relationships between each of the field emitters 206 to determine the location of magnetic field sensor 212 in tracking volume 210. Proceeding to block 706 “measure a rotating magnetic field with the sensor” a rotating magnetic field may be measured with the sensor. For example, electromagnetic sensor 312 may be used to measure a rotating magnetic field generated by marker 308 being rotated about a longitudinal axis of drive cable 310. In many embodiments, the marker 308 may comprise a magnet having poles aligned perpendicular to the longitudinal axis of the drive cable 310.

At block 708 “determine a location of a source of the rotating magnetic field relative to the sensor based on measurement of the rotating magnetic field” a location of a source of the rotating magnetic field relative to the sensor may be determined based on measurement of the rotating magnetic field. For example, a location of marker 308 (i.e., the source of the rotating magnetic field) may be determined relative to electromagnetic sensor 312 based on measurement of the rotating magnetic field. Accordingly, the location of a portion of delivery device 302 with respect to a portion of medical device 304 can be reliably and accurately determined.

Proceeding to block 710 “determine a location of the source of the rotating magnetic field in the tracking volume based on the location of the sensor in the tracking volume and the location of the source of the rotating magnetic field relative to the sensor” a location of the source of the rotating magnetic field in the tracking volume may be determined based on the location of the sensor in the tracking volume and the location of the source of the rotating magnetic field relative to the sensor. For example, controller 608 may determine the location of a distal end of medical device 604 in a tracking volume of tracking base 606 based on a location of a distal end of delivery device 602 in the tracking volume and a location of the distal end of medical device 604 relative to the distal end of delivery device 602.

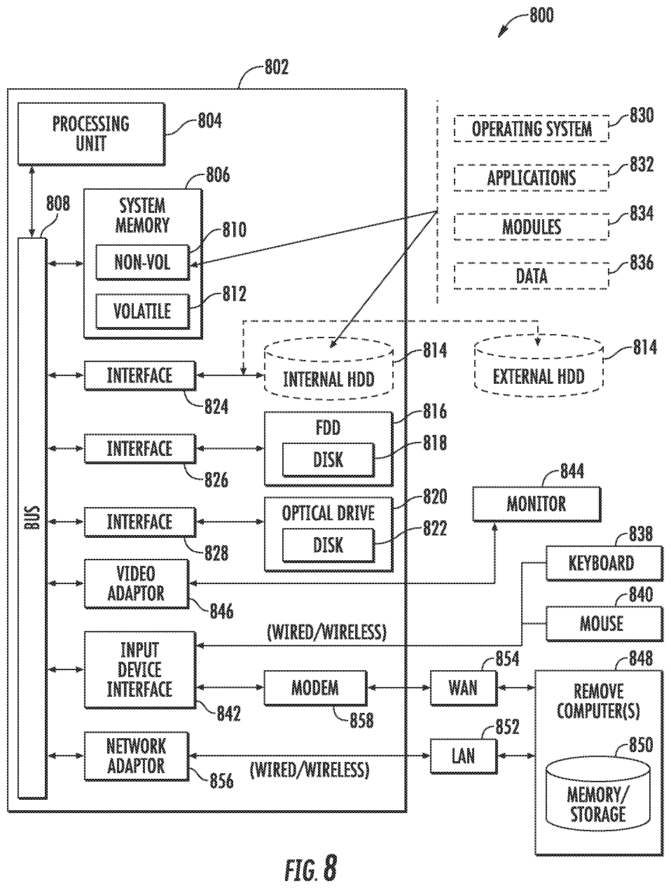

FIG. 8 illustrates a computing architecture 800 according to one or more embodiments disclosed hereby. The computing architecture 800 may be suitable for implementing various embodiments as previously described. In various embodiments, the computing architecture 800 may comprise or be implemented as part of an electronic device and/or medical device. In some embodiments, the computing architecture 800 may be representative, for example, of one or more components described hereby. In some embodiments, computing architecture 800 may be representative, for example, of a computing device that implements or utilizes one or more portions of components and/or techniques described hereby, such as controller 108, sensors 104, controller 608, logic circuitry 610, memory 612, I/O 614, and/or user interface 616. The embodiments are not limited in this context.

As used in various embodiments herein, the terms “system” and “component” and “module” can refer to a computer-related entity, either hardware, a combination of hardware and software, software, or software in execution, examples of which are provided by the exemplary computing architecture 800. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a controller 108 and the controller 108 can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 800 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 800.

As shown in FIG. 8, the computing architecture 800 comprises a processing unit 804, a system memory 806 and a system bus 808. The processing unit 804 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit 804.

The system bus 808 provides an interface for system components including, but not limited to, the system memory 806 to the processing unit 804. The system bus 808 can be any of several types of bus structure that may further interconnect to a memory bus (with or without a memory controller), a peripheral bus, and a local bus using any of a variety of commercially available bus architectures. Interface adapters may connect to the system bus 808 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 806 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory (e.g., one or more flash arrays), polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 8, the system memory 806 can include non-volatile memory 810 and/or volatile memory 812. In some embodiments, system memory 806 may include main memory. A basic input/output system (BIOS) can be stored in the non-volatile memory 810.

The computer 802 may include various types of computer-readable storage media in the form of one or more lower speed memory units, including an internal (or external) hard disk drive (HDD) 814, a magnetic floppy disk drive (FDD) 816 to read from or write to a removable magnetic disk 818, and an optical disk drive 820 to read from or write to a removable optical disk 822 (e.g., a CD-ROM or DVD). The HDD 814, FDD 816 and optical disk drive 820 can be connected to the system bus 808 by an HDD interface 824, an FDD interface 826 and an optical drive interface 828, respectively. The HDD interface 824 for external drive implementations can include at least one or both of Universal Serial Bus (USB) and Institute of Electrical and Electronics Engineers (IEEE) 994 interface technologies. In various embodiments, these types of memory may not be included in main memory or system memory.

The drives and associated computer-readable media provide volatile and/or nonvolatile storage of data, data structures, computer-executable instructions, and so forth. For example, a number of program modules can be stored in the drives and memory units 810, 812, including an operating system 830, one or more application programs 832, other program modules 834, and program data 836. In one embodiment, the one or more application programs 832, other program modules 834, and program data 836 can include or implement, for example, the various techniques, applications, and/or components described hereby.

A user can enter commands and information into the computer 802 through one or more wire/wireless input devices, for example, a keyboard 838 and a pointing device, such as a mouse 840. Other input devices may include sensors 104, sensors 114, tracking base 122, imaging sensor 306, electromagnetic sensor 312, microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 804 through an input device interface 842 that is coupled to the system bus 808 but can be connected by other interfaces such as a parallel port, IEEE 994 serial port, a game port, a USB port, an IR interface, and so forth.

A monitor 844 or other type of display device is also connected to the system bus 808 via an interface, such as a video adaptor 846. The monitor 844 may be internal or external to the computer 802. In addition to the monitor 844, a computer typically includes other peripheral output devices, such as speakers, printers, and so forth.

The computer 802 may operate in a networked environment using logical connections via wire and/or wireless communications to one or more remote computers, such as a remote computer 848. In various embodiments, one or more interactions described hereby may occur via the networked environment. The remote computer 848 can be a workstation, a server computer, a router, a personal computer, portable computer, microprocessor-based entertainment appliance, a peer device or other common network node, and typically includes many or all of the elements described relative to the computer 802, although, for purposes of brevity, only a memory/storage device 850 is illustrated. The logical connections depicted include wire/wireless connectivity to a local area network (LAN) 852 and/or larger networks, for example, a wide area network (WAN) 854. Such LAN and WAN networking environments are commonplace in offices and companies, and facilitate enterprise-wide computer networks, such as intranets, all of which may connect to a global communications network, for example, the Internet.

When used in a LAN networking environment, the computer 802 is connected to the LAN 852 through a wire and/or wireless communication network interface or adaptor 856. The adaptor 856 can facilitate wire and/or wireless communications to the LAN 852, which may also include a wireless access point disposed thereon for communicating with the wireless functionality of the adaptor 856.

When used in a WAN networking environment, the computer 802 can include a modem 858, or is connected to a communications server on the WAN 854 or has other means for establishing communications over the WAN 854, such as by way of the Internet. The modem 858, which can be internal or external and a wire and/or wireless device, connects to the system bus 808 via the input device interface 842. In a networked environment, program modules depicted relative to the computer 802, or portions thereof, can be stored in the remote memory/storage device 850. It will be appreciated that the network connections shown are exemplary and other means of establishing a communications link between the computers can be used.

The computer 802 is operable to communicate with wire and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Various embodiments may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor (e.g., logic circuitry), which when read by a machine causes the machine to fabricate logic to perform the techniques described hereby. Such representations, known as “IP cores” may be stored on a tangible, machine readable medium and supplied to various customers or manufacturing facilities to load into the fabrication machines that actually make the logic or processor. Some embodiments may be implemented, for example, using a machine-readable medium or article which may store an instruction or a set of instructions that, if executed by a machine (e.g., logic circuitry), may cause the machine to perform a method and/or operation in accordance with the embodiments. Such a machine may include, for example, any suitable processing platform, computing platform, computing device, processing device, computing system, processing system, computer, processor, logic circuitry, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

All of the devices and/or methods disclosed and claimed hereby can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method disclosed hereby without departing from the concept, spirit and scope of the disclosure. All such similar substitutes and modifications apparent to those skilled in the art are deemed to be within the spirit, scope and concept of the disclosure as defined by the appended claims.

1. A system, comprising:
a medical device comprising a proximal end, a distal end, and a lumen extending between the proximal and distal ends;
a drive cable having a longitudinal axis, the drive cable disposed in the lumen of the medical device and configured to rotate about the longitudinal axis;
a marker attached to the drive cable, wherein the marker is rotatable by the drive cable; and
a sensor configured to detect the marker rotating about the longitudinal axis.

2. The system of claim 1, wherein the marker comprises a magnet and poles of the magnet are aligned perpendicular to the longitudinal axis.

3. The system of claim 1, comprising an imaging sensor attached to the drive cable, wherein the imaging sensor is rotatable by the drive cable.

4. The system of claim 1, comprising a delivery device comprising a proximal end, a distal end, and a working channel extending between the proximal and distal ends, wherein the sensor is disposed in the delivery device and the medical device is disposed in the working channel.

5. The system of claim 1, wherein the marker comprises a magnet and the magnet is radially symmetric with the longitudinal axis of the drive cable.

6. The system of claim 1, wherein the sensor is disposed in a tracking base, the tracking base comprising a plurality of magnetic field emitters for generating a plurality of reference magnetic fields.

7. The system of claim 1, wherein the sensor comprises a 6 degrees of freedom (DOF) tunnel-magnetoresistance (TMR) sensor.

8. An apparatus, comprising:
a delivery device comprising an elongate member with a proximal end, a distal end, and a working channel extending between the proximal and distal ends;
a first sensor disposed proximate the distal end of the elongate member, the first sensor configured to localize a portion of the delivery device in six degrees of freedom;
a second sensor disposed proximate the working channel, the second sensor configured to measure one or more characteristics of a medical device disposed in the working channel of the delivery device to determine a position of the medical device relative to the delivery device.

9. The apparatus of claim 8, wherein the position of the medical device relative to the delivery device comprises one or more of a linear translation and a rotational translation.

10. The apparatus of claim 8, wherein the one or more characteristics comprise one or more markers encoded with the position of the medical device relative to the delivery device.

11. The apparatus of claim 10, wherein the one or more markers comprise one or more barcodes or one or more quick response (QR) codes.

12. The apparatus of claim 10, wherein the one or more markers comprise a plurality of markers disposed along a portion of the medical device, and wherein each of the plurality of markers indicate one or more of a linear translation and a rotational translation of the medical device.

13. The apparatus of claim 8, wherein the one or more characteristics comprise changes in an outer surface of the medical device.

14. The apparatus of claim 13, wherein the second sensor emits and detects infrared or near-infrared light bounced off the outer surface of the medical device to determine a position of the medical device relative to the delivery device.

15. The apparatus of claim 28, comprising:
a processor communicatively coupled to the first and second sensors; and
a memory communicatively coupled to the processor, the memory comprising instructions that when executed by the processor cause the processor to determine a location of the medical device based on localization of a portion of the delivery device in six degrees of freedom and the position of the medical device relative to the delivery device.

16. The apparatus of claim 15, the memory comprising instructions that when executed by the processor cause the processor to:
measure a plurality of reference magnetic fields with the first sensor; and
localize the portion of the delivery device in six degrees of freedom based on measurement of the plurality of reference magnetic fields.

17. A computer-implemented method, comprising:
measuring a plurality of reference magnetic fields with a sensor;
determining a location of the sensor in a tracking volume based on measurement of the plurality of reference magnetic fields;
measuring a rotating magnetic field with the sensor;
determining a location of a source of the rotating magnetic field relative to the sensor based on measurement of the rotating magnetic field; and
determining a location of the source of the rotating magnetic field in the tracking volume based on the location of the sensor in the tracking volume and the location of the source of the rotating magnetic field relative to the sensor.

18. The computer-implemented method of claim 17, comprising rotating a drive cable coupled to a magnet to generate the rotating magnetic field.

19. The computer-implemented method of claim 17, comprising:
generating an image with an imaging sensor;
adding the image to a mapping of an interior of a body based on the location of the source of the rotating magnetic field in the tracking volume.

20. The computer-implemented method of claim 19, comprising rotating a drive cable coupled to the imaging sensor and a magnet to generate the rotating magnetic field, and wherein the image comprises a radial ultrasound image.
